(19) United States
(12) Patent Application Publication
(10) Pub. No.: US 2004/0018220 A1
(43) Pub. Date: Jan. 29, 2004

(54) Aqueous compositions for facial cosmetics

(75) Inventors: Win L. Chiou, Burr Ridge, IL (US); Linda L. Chiou, Burr Ridge, IL (US)

Correspondence Address: MERCHANT & GOULD PC, P.O. BOX 2903, MINNEAPOLIS, MN, 55402-0903, US

(73) Assignee: Chiou Consulting, Inc.

(21) Appl. No.: 10/608,714

(22) Filed: Jun. 23, 2003

Related U.S. Application Data
(63) Continuation-in-part of application No. 10/206,076, filed on Jul. 26, 2002, now Pat. No. 6,616,923.

(51) Int. Cl. A61K 7/00

(52) U.S. Cl. 424/401

(57) ABSTRACT

The present invention describes a highly versatile and effective facial composition containing two very simple human endogenous compounds, glycerin or any other nonvolatile, pharmacologically acceptable, slowly absorbed, water-miscible, liquid organic substance and water, which, when applied topically to facial skin in high concentrations, is surprisingly effective for achieving facial skin improvements. The effect of cosmetic improvements lasts up to 24 hours after one single application of the composition. The novel, transparent or semi-transparent, unconventional aqueous composition is especially useful for postmenopausal women.

### CROSS-REFERENCE TO RELATED APPLICATION

This application is a continuation-in-part of U.S. Ser. No. 10/206,076, filed Jul. 26, 2002, now allowed.

### FIELD OF THE INVENTION

The present invention relates to improving facial skin with an aqueous composition containing one or more non-volatile, slowly absorbed, water-miscible, liquid organic substances.

### BACKGROUND

There are a number of facial skin care products on the market, most of which are in the form of cream, ointment, lotion, gel or emulsion. The majority of these products are not miscible with water and generally contain numerous ingredients, e.g., about 20 to 40 different ingredients. For example, after a gentle mixing with about 10 parts of water, there is a clear separation between water and most of the product. One problem with these complex facial skin care products is that they are relatively difficult to prepare, often requiring special skills and facilities. Additionally, due to their numerous ingredients and methods of preparation, the products are often very expensive.

Current facial skin care products are also often relatively unstable in physical shape and form as well as in chemical composition. This instability, which includes thermal instability, may have a drastic effect on the usefulness of a product which has been exposed to variable temperatures, e.g., ranging from about 0° to about 40° C., during shipping, storage, or use. Additionally, many of the ingredients present in commercial facial skin care products may break down or degrade quickly upon contact with the environment.

Furthermore, most of the ingredients, such as mineral oil, waxes, lanolins, silicones, surfactants, dyes, plant extracts, proteins and preservatives, in commercial facial skin care products are foreign to the human body, i.e., xenobiotics, and may often cause allergic reactions in those who use these products. Those with sensitive skin may find many of the ingredients in commercial facial skin care products harsh and thus, unacceptable for use.

In facial cosmetics and skin care glycerin is commonly used as a solvent, sweetener and humectant. Sugars and starches are also commonly regarded as humectants. Glycerin helps to prevent creams from drying out (“Make Your Own Cosmetics,” Aurum Press Ltd., London, 1997, p. 128). It appears that the percutaneous absorption of pure glycerin is commonly assumed to be negligible in humans. In many commercial complex water-immiscible ointments, creams and lotions containing glycerin, use of liposomes or other advanced absorption-enhancing methods is apparently employed in order to facilitate instant or rapid absorption of product ingredients into skin cells. This can be clearly illustrated by the example of Neutrogena's Body Moisturizer. Therefore, it seems not surprising that this and many other similar products may leave an apparently dry film on the skin surface shortly (usually within about 15 to 30 minutes) after application (see Example I). Interestingly, glycerin is commonly regarded as an inactive ingredient in topical products used to reduce fine lines, wrinkles, age spots, and to increase firmness and smoothness; this notion is explicitly shown on the label of Neutrogena's ANTI-WRINKLE CREAM. A major aim of the present invention is to describe several surprising, highly effective new uses of glycerin and other water-miscible slowly absorbed liquid organic substances.

Thus, there exists a need for facial skin care methods and formulations that are hypo-allergenic, natural, inexpensive, easy to prepare, and highly stable, as well as highly effective in producing facial cosmetic benefits or improvements.

### SUMMARY OF THE INVENTION

Unlike the complex facial skin care products described above and currently on the market, the present invention provides for a surprisingly simple, safe, water-miscible, transparent/semi-transparent, hypo-allergenic, inexpensive, easy to use, easy to prepare, stable, highly efficient and effective composition for improving the facial skin of humans. This unconventional and yet simple approach for facial skin care includes a method of using an aqueous composition, i.e., composition or mixture containing (in terms of percentage of total weight) of one or more nonvolatile, slowly absorbed, water-miscible, liquid organic substance(s) and water in the absence or presence of other skin nutrients or skin-improving ingredients for achieving facial cosmetic improvements. For example, a single application of the aqueous, facial cosmetic composition of the present invention results in several beneficial cosmetic effects which last for a long period of time, for example, for at least about 4 to 18 hours (see Example I). The slowly absorbed, nonvolatile, liquid organic substance and water serves as a very simple, inexpensive, highly effective, protective, mechanical barrier or coating on the facial skin surface which prevents or minimizes evaporation of body water from the surface of the facial skin. Additionally, the facial cosmetic composition of the invention is a highly effective moisturizing agent. Since a liquid organic substance of the present invention, for example, glycerin, is a human endogenous substance, its combination with water provides a composition which is practically free of allergic reactions and extremely safe.

One feature of the present invention is the unexpected discovery that water can be used as a highly effective and efficacious anti-wrinkle and/or anti-line agent. In one embodiment, water is continuously and constantly delivered to the facial skin (excluding chapped or burned skin) for an extended period of time, e.g., for about 4 to 20 about 18 hours, preferably for about 8 to about 24 hours after a single application of a thin layer of a transparent aqueous composition containing water and one or more nonvolatile, slowly absorbed, water miscible, liquid organic substances. The extended effect of the applied water on the facial skin is analogous to-the “morning dew” phenomenon (refreshing and revitalizing of the withered leaves and flowers) observed on plants or flowers in the early morning following a light rain or foggy night; watering of withering domestic plants or flowers has been repeatedly observed to result in a reappearance of fresh, shin y, dewy, healthy, wrinkle-free smooth leaves or flowers the next day. Therefore, continuous/daily use of the aqueous composition of the present invention serves as a prophylactic treatment to prevent or minimize the formation of wrinkles and/or lines on the facial skin (see Examples II and III). The beneficial cosmetic effects of the present invention is especially useful on post-menopausal women (see Example III).

A particular embodiment of the present invention is a method of using a facial skin care composition containing glycerin and water in the absence or presence of other skin nutrients or skin-improving ingredients, to provide effective improvement of the facial skin of humans. For example, the present invention provides methods for treating or ameliorating lines, wrinkles, and dark spots on the skin. Additionally, the methods of the present invention are capable of treating or ameliorating dry skin and signs of aging. The methods of the present invention are also capable of improving the elasticity, firmness, smoothness, and appearance of skin. The other skin nutrients to be used may include, for example, vitamins, amino acids, minerals, electrolytes, glucose and skin lipids such as ceramide and cholesterol. A preservative and fragrance may also be added. A sunscreening agent may also be employed.

The present invention provides a method for cosmetically improving facial skin of humans by administering to the facial skin surface an aqueous composition containing an effective amount of one or more nonvolatile (not evaporating or evaporating less than about 10% in 4 hours at normal temperatures and pressures with boiling points preferably higher than about 150° C.), pharmacologically acceptable (not irritating to the skin and causing no adverse effects after being administered and/or absorbed through the skin to the circulating blood), slowly absorbed (most absorption occurring for about 4 to about 18 hours, preferably from about 8 to about 24 hours, and more than about 5% to about 10% absorption occurring after 4 hours), water-miscible (capable of being mixed homogeneously with water practically at any ratio) liquid organic substances (e.g., liquid substances with organic chemical structures). The present invention performs much more effectively, up to about 48-fold better, than conventional, complex and expensive products (see Examples I, IV and V for moist, dewy and shiny effects). The amount of the liquid organic substance in the final product may range from about 4% to about 99.9% by weight, and a viscosity-enhancing or gelling agent may be included mainly for the ease and convenience of the application of the product (see Examples IV and V). The liquid organic substances useful in this invention include, but are not limited to, glycerin, triethylene glycol, diglycerol, glyceryl monoacetate, glyceryl diacetate and polyethylene glycols (PEG) such a PEG 200, 300, 400 and 600, and other effective glycol or glyceryl derivatives.

The invention also provides a method for treating wrinkles on facial skin of a human by administering to the facial skin surface an aqueous composition which includes an effective amount of one or more nonvolatile, slowly absorbed, water-miscible liquid organic substances.

The invention also provides a method for treating age or dark spots on facial skin of a human by administering to the facial skin surface an aqueous composition which includes an effective amount of one or more nonvolatile, slowly absorbed, water-miscible liquid organic substances.

The invention also provides a method for treating lines on facial skin of a human by administering to the facial skin surface an aqueous composition which includes an effective amount of one or more nonvolatile, slowly absorbed, water-miscible liquid organic substances.

The invention also provides a method for treating the signs of aging on facial skin of a human by administering to the facial skin surface an aqueous composition which includes an effective amount of one or more nonvolatile, slowly absorbed, water-miscible liquid organic substances.

The invention also provides a method for increasing the elasticity, firmness and smoothness of facial skin of a human by administering to the facial skin surface an aqueous composition which includes an effective amount of one or more nonvolatile, slowly absorbed, water-miscible liquid organic substances.

The invention also provides a method for improving the texture or appearance of facial skin of a human comprising administering to the facial skin surface an aqueous composition of an effective amount of one or more nonvolatile, slowly absorbed, water-miscible liquid organic substances.

The invention further provides a method for promoting shiny, moist facial skin surface lasting for at least about 4 hours and up to about 24 hours (see Examples I, IV and V) by administering to the facial skin surface of a human an aqueous composition including an effective amount of one or more nonvolatile, slowly absorbed, water-miscible liquid organic substances.

The invention also provides a method of providing nutrients (e.g., large amounts of glycerin and water) to the facial skin of a human by administering to the facial skin surface an aqueous composition containing an effective amount of one or more nonvolatile, slowly absorbed, water-miscible liquid organic substances (e.g. glycerin).

The present invention provides a simple and novel method of enhancing the absorption of poorly permeable compounds such as glucose, and amino acids by increasing their contact time on the skin surface.

Additional ingredients, such as, for example, vitamins, minerals, amino acids, electrolytes, skin lipids, glucose, antioxidants and sunscreening agents, known to improve or treat facial skin when administered orally or topically, can also be simply and inexpensively added to the above aqueous composition for further improving the cosmetic or skin-care effects.

The invention also provides a surprisingly very simple and extremely safe method for inhibiting the growth and/or killing of microorganisms on the facial surface for a prolonged period of time (at least about 4 hours, preferably about 8 hours to about 24 hours) comprising administering to the facial skin surface an aqueous composition of an effective amount of one or more nonvolatile, slowly absorbed, water-miscible liquid organic substances (e.g., glycerin and PEG 400).

The aqueous composition of the present invention may be applied to facial skin as often as appropriate or desired. Preferably, two facial applications a day (one in the morning and one prior to bedtime) ensures practically continuous cosmetic or skin-care benefits throughout the day and evening.

### DETAILED DESCRIPTION OF THE INVENTION

As used herein, the phrase “facial improvements” includes a reduction in the signs of aging including, but not limited to, a reduction in wrinkles, fine lines, and/or age or dark spots. The phrase “facial improvements” also refers to an increase in skin elasticity, softness, smoothness, dewiness, shininess, and/or firmness. “Facial improvements” further refers to moisturizing of facial skin, treatment of wrinkles, fine lines, dark spots and/or the signs of aging, but does not include cleaning of facial skin. It may also include inhibition and/or killing of microorganisms on the facial surface.

As used herein, the phrase “facial cosmetics” refers generally to products used for facial improvement commonly sold in professional cosmetic stores.

As used herein the term “treat” includes treating, preventing, ameliorating, or inhibiting a skin condition, including age or dark spots, fine lines, wrinkles, signs of aging, or generally resulting in at least one facial improvement, including an increase in skin elasticity, softness, smoothness, dewiness, shininess, firmness, moisture content and fewer lines, wrinkles and/or dark spots. It may also include inhibition and/or killing of microorganisms.

As used herein, the phrase “aqueous composition” refers to aqueous liquid mixtures that are not water-immiscible creams, ointments or lotions, and are, preferably, transparent or semi-transparent. For the ease of application an appropriate amount, e.g., from about 0.1% to about 8% of a suitable viscosity-enhancing or gelling agent can be incorporated into the mixture (see Examples IV and V); a higher percent of the viscosity-enhancing or gelling agent may be used if the agent chosen is not very effective compared for example with carbomer.

As used herein, the phrase “nonvolatile, slowly absorbed, water miscible, liquid organic substance” refers to liquid organic substances with high boiling points, such as preferably 150° C. or higher, that will not significantly (for example, less than 10%) evaporate on the surface of facial skin within 4 hours and can be homogeneously mixed with water practically at any ratio. Such substances include, but are not limited to, glycerin, diglycerol, glyceryl monoacetate, glyceryl diacetate, triethylene glycol, polyethylene glycols, such as PEG 200, 300, 400 and 600, and other polyethylene glycols with different mean molecular weights which are commercially available; other effective glycol or glyceryl derivatives are also included. Preferably, most of the liquid organic substance is slowly absorbed by the facial skin surface for at least about 4 hours to about 18 hours, preferably from about 8 to about 24 hours.

As used herein, “slowly absorbed” is qualitatively defined as a significant portion (e.g. about 5% to about 20%) of the water miscible, liquid organic substance being able to physically remain on the facial skin surface for a long period of time. This is determined by the presence of a layer of the aqueous composition or pure liquid organic substance remaining on the facial skin through a “moist” feeling or chemical assay. For example, since glycerin will not evaporate at 37° C., its disappearance from the skin surface indicates its absorption through the facial skin. An aqueous composition containing 51% propylene glycol, a nonvolatile, water miscible, liquid organic substance, virtually disappears from the facial surface in less than two hours, while a composition containing 56% glycerin or 53% polyethylene glycol 400 or 600 remains on the facial skin for at least about 4 hours and up to about 24 hours.

Preferred compositions useful for carrying out the methods of the invention contain, in terms of percentage of the total weight, one or more water-miscible, liquid organic substances and water, including an aqueous composition of glycerin and water, more preferably with the addition of other skin nutrients or skin-improving ingredients (see Example V).

Due to its very high boiling point (290° C.; The Merck Index, 12^{th} edition, S. Budavari et al., Merck & Co., Inc., Whitehouse Station, N.J. (1996)), glycerin from an aqueous glycerin and water composition applied as a thin layer to the facial skin will not evaporate; instead, it will be very slowly absorbed into the skin over a period of from about 8 hours to about 24 hours (see Example I). Since applied glycerin can be substantially or completely absorbed, although slowly, into the percutaneous tissue, a substantial amount of water can be simultaneously absorbed by the facial skin through passive diffusion of the hydrated glycerin (water attached to the glycerin molecule). Additionally, the high concentration of water in an aqueous glycerin can be slowly absorbed into the facial skin through a concentration-gradient-driven diffusion process. Furthermore, a high concentration of glycerin on the surface of the facial skin will form a protective, mechanical layer to prevent or minimize loss of body water due to evaporation from the facial skin for a long period time as above described. The absorbed water and the prevention of water evaporation plays a very important role in maintaining the integrity and normal function of the facial skin. The protective glycerin may also enhance the percutaneous permeability of water.

Additionally, glycerin can effectively retain water from an aqueous glycerin solution applied on the facial skin surface; this was estimated to be about 10% to about 25% based on in vitro studies. Thus, an aqueous cosmetic product containing an effective amount of glycerin and/or another water-miscible, slowly absorbed liquid organic substance and water applied to the facial skin surface can serve as a powerful, constant moisturizing vehicle and thereby make the facial skin instantly, as well as for many hours (see above) shiny, moist and dewy (see Example I). These results are in sharp contrast with conventional commercial facial cosmetic products where the moist effects on the skin surface of the product last only about 0.5 to about 2 hours (see Example I; the difference is up to about 48-fold). In one embodiment of the present invention, the applied water serves as a highly effective anti-wrinkle and/or anti-line agent.

Additionally, since glycerin can be metabolized into glucose, glycerol-3-phosphate, pyruvic acid, water and carbon dioxide with the release of energy (Medical Physiology, (2000)), the large amount of glycerin used in one embodiment of the present invention can serve as a nutrient and an energy source for maintenance and improvement of facial skin cells for many hours, as above described, by providing the aforementioned metabolic products.

An appropriate combination of the two active ingredients, a water-miscible, slowly absorbed, liquid organic substance, such as glycerin, and water, can be used for application to the facial skin for moisturization and for treatment of dry facial skin (facial skin with reduced moisture as compared to normal facial skin), lines and wrinkles (a line or crease in the facial skin, such as those caused by sun exposure or old age) and for treatment of dark spots or age spots (facial skin disorder seen with aging or sun exposure) where there are flat patches of increased pigmentation on the facial skin.

Additionally, the above two active ingredients can be administered to the facial skin to reduce the signs of aging (gradual changes in the structure, function and appearance of facial skin, such as drier facial skin, wrinkles and age spots, that occur with the passage of time and do not result from disease, accident or wound). They can also be used in a method for increasing the elasticity of facial skin (the facial skin's ability to stretch) and improving the texture (smoothness or firmness) and appearance (plump, shiny, whiter, dewy, smooth and fresh) of facial skin.

Also, the glycerin and/or another water-miscible liquid organic substance that have been absorbed by the facial skin can function as moisturizing agents underneath the facial skin surface and improve the texture and appearance or quality of the facial skin.

The aqueous composition of the present invention can contain various effective amounts of a water-miscible liquid organic substance or a mixture of water-miscible liquid organic substances. The amount may range from about 4% to about 99.9% or about 10% to about 99.9% by weight. Preferably, the amount may range from about 7% to about 90% or from about 10% to about 85%, more preferably from about 20% to about 70%, and most preferably from about 25% to about 65%. Application of a pure liquid organic substance or a mixture of pure organic liquid substances, although not most desirable, onto the facial skin may also be expected to produce similar skin-improving benefits discussed above (see Example IV). This is because the liquid organic substance is able to absorb water from the air and also mix with the minute moisture present naturally on the skin. The absorption of water from the air was estimated to be from about 10% to about 25% of the weight of glycerin. Also, the pure liquid organic substance may be applied to a highly wet skin immediately or shortly after washing of face with the water. Alternatively, an effective amount of the pure liquid can be applied first to the skin followed by the application of a small amount of water.

The aqueous composition can be applied to the facial skin by any appropriate method, such as a spray bottle, a droplet bottle, a bottle with a pump, a soaked cotton ball, cloth or pad, as well as a collapsible tube or a bottle for a gel. Although the aqueous composition is primarily useful for facial application, the composition may also be applied to any other part of the human body where skin improvement benefits are desired. It is to be noted that as expected, the rate of absorption of liquid organic substances may vary significantly with type and location of skin on the body. Placing a soaked cloth on the facial surface for a few hours is expected to increase the beneficial effect of the aqueous composition.

The present method differs from commercially available facial cosmetic and facial skin-care products. First, in most commercially available facial skin-care products, glycerin or other water-miscible organic liquid substances are only present as very minor components in the products and the presence and function of water is not emphasized. For example, the lack of recognition of the great importance of glycerin and water in facial cosmetics is clearly illustrated on the label of a cream product marketed by Neutrogena Corporation (Los Angeles, Calif.). The label of Neutrogena's 26-ingredient Anti-Wrinkle Cream with SPF 15 lists glycerin and water as inactive ingredients. Additionally, many widely used water-immiscible facial cosmetic and facial skin-care products, such as ointments, creams, gels and lotions, typically contain some 15 to 30 xenobiotics (foreign to the human body). When applied to the facial skin surface, water in these commercial products often evaporates very rapidly within about 30 to 60 minutes and a “dry film” is left on the surface. This is in sharp contrast with the water-miscible, transparent or semi-transparent (see Example V) aqueous compositions of the present invention. Surprisingly, after just one application of a 56% aqueous glycerin composition (56% AGS) or a 53% aqueous polyethylene glycol 400 composition (53% APS) or other aqueous compositions containing different amounts of glycerin (see Examples IV and V) to the facial skin, the facial skin feels moist for many hours (see Example I). Thus, the moisturizing efficiency and effectiveness on the surface of the facial skin and improvement in the appearance of the facial skin from the present invention is about up to 48 fold better over some currently available commercial products (see Example I).

Use of the aqueous facial composition twice daily can virtually produce facial improvements and/or benefits for the entire day.

The aqueous glycerin composition of the invention is much easier to prepare, for example, by merely a one-step mixing of the liquid organic substance and water. The resulting composition is more stable (both chemically and physically stable) over a wide range of temperatures than commercially available creams, ointments, emulsions, gels and lotions. Thus, in addition to the advantageous cosmetic benefits provided by the methods of the invention, the use of an aqueous composition of the present invention provides advantages in cost, ease of preparation, and ease of product handling and storage compared to commercial water-immiscible creams, ointments, emulsions, gels, and lotions. For aqueous compositions containing additional skin-improving ingredients and a viscosity-enhancing or gelling agent such as one shown in Example V, preparation can be easily achieved by first mixing the water-soluble ingredients in the water and mixing the glycerin-soluble ingredients in the glycerin prior to final mixing of the two mixtures. Heating can be used to facilitate dissolution of ingredients. Also, the presence of limited amounts of very fine particles evenly suspended in a viscous aqueous composition or aqueous gel is also acceptable in the present invention. Also, a co-solvent such as propylene glycol or butylene glycol and a surfactant may also be used if needed for solubilization.

The invention also provides a surprisingly very simple and extremely safe method for inhibiting the growth and/or killing of microorganisms on the facial surface for a prolonged period of time (at least about 4 hours, preferably about 8 hours to about 24 hours) comprising administering to the facial skin surface an aqueous composition containing an effective amount of one or more nonvolatile, slowly absorbed, water-miscible liquid organic substances, e.g., glycerin and water. Microorganisms including virus and fungus can be effectively inhibited and killed by a high concentration of glycerin or polyethylene glycol 400 (see Example VI). When a diluted aqueous glycerin mixture, containing, for example, about 10% to about 70% by weight of glycerin, is applied to the facial surface, in addition to slow percutaneous absorption, some water will evaporate quickly (probably in a few minutes to about 30 minutes) and the glycerin content on the skin surface is expected to approach about 75 to about 85% and remain at this level until virtually all of the glycerin is absorbed into the skin. Such a very high glycerin solution is expected to inhibit or kill all the microorganisms on the surface of skin. A microorganism-free or sterile skin surface may be desirable for minimization or prevention of facial infection in normal subjects and highly desirable for some individuals, for example, with acne or a tendency to have acne in view of the fact that topical antibiotics, such as tetracycline and erythromycin, requiring a physician's prescription are commonly employed to treat acne. Thus, the present invention can provide a very simple and safe method for prophylaxis and treatment of acne. Although most facial cosmetic products contain preservatives such as methylparaben and propylparaben, these preservatives are known to be rapidly absorbed into the skin, and cannot produce a very prolonged antibacterial effect on the skin surface as invented here. Also, some natural or synthetic antimicrobial ingredients or extracts from plants such as tea can be incorporated into the aqueous composition to augment the antimicrobial action.

The present invention will now be illustrated by the following non-limiting examples. The percentages of ingredients, unless otherwise indicated above or hereinafter, are by weight.

### EXAMPLE I

Facial Cosmetic Effects Observed After a Single Application of Aqueous Glycerin Composition (AGS), Aqueous Polyethylene Glycol 400 Composition (APS) or Commercial Facial Cosmetics

A 56% aqueous glycerin composition (56% AGS by weight; e.g., 100 ml glycerin mixed with 100 ml distilled water) was prepared. This composition was applied in the morning as a thin layer on the left-side of the face of two human subjects. A commercial cream, Elizabeth Arden—Ceramide Time Complex Moisture Cream that contains 37 ingredients, was applied to the right-side of the face of the same two subjects. Both sides of the face immediately appeared shiny, moist and dewy. However, the right-side of the face lost the shiny, moist and dewy appearance in both subjects within about two hours after application of the cream. The left-side of the face maintained its shiny, moist and dewy appearance for about 12 to 18 hours in both subjects. These results clearly demonstrate that a simple 56% AGS is much more effective (almost 10 fold) in producing a shiny, moist and dewy appearance than the much more expensive commercial cosmetic cream. The above superiority of the 56% AGS was demonstrated repeatedly. In some areas the moist feeling lasted about 24 hours. No adverse effects were reported.

The performance of the 56% AGS was also similarly compared in two human subjects with another commercial facial cosmetic cream, Loreal Plenitude—Turning Point (Loreal Retail Division of Costar Inc., New York). Loreal Plenitude—Turning Point contains 20 different ingredients. When compared, the shiny, moist and dewy appearance of the subjects facial skin lasted for about 15 hours after one application of the 56% AGS, while Loreal Plenitude—Turning Point lasted only about one hour. Short acting effects, lasting for only about 30 minutes were also demonstrated with other commercial facial cosmetic products including Neutrogena's Body Moisturizer. Therefore, the difference in the length of time for the above cosmetic effect is up to about 48-fold between 56% AGS and commercial cosmetic products.

A 53% Aqueous Polyethylene Glycol 400 Composition (53% APS by weight) was also similarly prepared. This composition was applied in the morning to two human subjects. Effects similar to those observed with the above 56% AGS were observed. No adverse effects were reported.

### EXAMPLE II

Facial Cosmetic Effects after Two Months of Application of the 56% Aqueous Glycerin Composition (56% AGS)

The 56% AGS was applied twice daily (once in the morning and once before bedtime) for about two months to the right-side face of two human adults, one male and one female. The right side and left side of the subject's faces were compared. In the male subject, the right side was determined to be more tender, shiny, fuller, and firmer. Additionally, the lines beneath the right eye were also found to be smaller, shallower and lighter. Similarly, the right side of the face of the female subject was determined to be more shiny, more tender, “whiter”, and practically free of dark spots as compared with the left side of the face of the female subject. The above discovery clearly demonstrated the high effectiveness of the simple 56% AGS in achieving desirable facial cosmetic improvements.

### EXAMPLE III

Surprising Facial Cosmetic Results after Five Years of Application of the 56% Aqueous Glycerin Composition (56% AGS) to a Post-menopausal Woman

A female subject used various brand-name facial cosmetic products (creams and lotions) twice daily for about three decades. At age 55 and at the beginning of menopause, she started to use 56% AGS twice daily (once in the morning and once prior to bedtime). At age 60, both sides of her face were determined to look younger and much whiter, more shiny, more elastic and firmer than about five to eight years ago. The above results clearly demonstrated the very dramatic effect of such a simple glycerin and water composition in improvement of facial skin appearance and on reversing the signs of aging after long-term application to a post-menopause woman. The test subject did not receive any replacement hormone therapy during the entire study. The present study may be particularly significant because dryness of skin and the related effects are known to occur in woman after menopause.

### EXAMPLE IV

Testing of Glycerin U.S.P. and Other Strengths of Aqueous Glycerin Solutions and Gels

The following were tested for shiny, moist, softening and dewy effects after a single application to a human subject: Glycerin U.S.P., 12%, 78%, and 92% (w/w) aqueous glycerin solutions, 12% and 56% aqueous glycerin gels. The gels were prepared with a 2% gelling agent, carbomer. As expected, results similar to those described in Example I were obtained. For the 100% glycerin U.S.P., better feeling (reduced stickiness) was obtained when it was applied to a wetted facial surface. For the 12% solution or gel a much higher amount was needed to achieve the same effect obtained by a 56% glycerin solution or viscous mixture as shown in Example V. It appears that the length and magnitude of effects are approximately proportional to the total amount of glycerin applied to the facial surface.

### EXAMPLE V

A Cosmetic/Skincare Formulation

| Ingredients | Percentage by Weight |
|---|---|
| Distilled water | 50.0 |
| Glycerin | 44.0 |
| Essential amino acids | 0.6 |
| Water-soluble vitamins | 0.2 |
| Lipid-soluble vitamins | 0.1 |
| Essential electrolytes and minerals | 2.0 |
| Skin lipids | 0.1 |
| Glucose | 1.5 |
| Carbomer for increasing viscosity | 1.0 |
| Sodium hydroxide for pH adjustment | 0.5 |
| Total | 100.0 |

Viscous semi-transparent liquid mixtures with a formulation similar to the above Example were prepared and tested twice daily on two subjects for at least seven months. Significant skin-care improving effects were obtained without any adverse reactions reported. Other suitable viscosity or gelling agents such as sodium carboxymethylcellulose can also be used. A bottle with a pump was used for convenient delivery of the product.

### EXAMPLE VI

Antimicrobial Activity of Glycerin and Polyethylene Glycol 400

A viscous liquid mixture similar to Example V and containing about 55% by weight of glycerin was prepared under non-sterile conditions and repeatedly exposed to ordinary room air over a 3-month period. Such a nutrient-containing mixture was determined to be sterile. Also, addition of a large quantity of saliva samples from two subjects resulted in no growth of microorganisms after storage for weeks at room temperature. A 53% polyethylene glycol 400 aqueous solution was also found to effectively inhibit the growth of microorganisms contaminated with human saliva and room air for weeks. A 53% polyethylene glycol solution was also found to effectively inhibit any growth of microorganisms in meat soup or apple juice. The reduction of free water in concentrated water-miscible-organic-solvent aqueous mixtures may be partly contributing to their antimicrobial activity.

It is to be understood that the above descriptions are intended to be illustrative, and not restrictive. For example, when necessary, other water-miscible high boiling-point liquids like propylene glycol, polyethylene glycol 400 and polyethylene glycol 600 may also be included in the glycerin and water formulation. Many other equivalents will be apparent to those of skill in the art upon reading and understanding the above description. Additionally, one skilled in the art will be able to ascertain, with no more than routine experimentation, many equivalents to the specific embodiments described herein. These equivalents are intended to be encompassed by the following claims.

What is claimed is:

1. A method for ameliorating facial wrinkles comprising administering to facial skin of a human an aqueous composition, that is not a water-immiscible cream, ointment or lotion, consisting essentially of an effective amount of one or more nonvolatile, slowly absorbed, water-miscible, liquid organic substances, and water, wherein the liquid organic substance is present from about 4% to about 99.9% by weight, and wherein the liquid organic substance is slowly absorbed into the facial skin over a period of time for at least about 4 hours.

2. The method of claim 1, wherein the liquid organic substance is slowly absorbed into the facial skin over a period of time from about 8 hours to about 24 hours.

3. The method of claim 1, wherein the liquid organic substance is present from about 20% to about 70% by weight or from about 10% to about 85% by weight.

4. The method of claim 1, wherein the aqueous composition consists essentially of glycerin and water.

5. The method of claim 1, wherein the liquid organic substance is glycerin, diglycerol, a polyethylene glycol, an effective glyceryl derivative, an effective glycol derivative, or a mixture thereof.

6. The method of claim 5, wherein the polyethylene glycol is polyethylene glycol 200, polyethylene glycol 300, polyethylene glycol 400 or polyethylene glycol 600.

7. The method of claim 1, wherein the aqueous composition further comprises a viscosity-enhancing or gelling agent at a concentration of about 0.1% to about 8% by weight.

8. A method for ameliorating facial lines on skin comprising administering to facial skin of a human an aqueous composition, that is not a water-immiscible cream, ointment or lotion, consisting essentially of an effective amount of one or more nonvolatile, slowly absorbed, water-miscible liquid organic substances, and water, wherein the liquid organic substance is present from about 4% to about 99.9% by weight, and wherein the liquid organic substance is slowly absorbed into the facial skin over a period of time for at least about 4 hours.

9. The method of claim 8, wherein the liquid organic substance is slowly absorbed into the facial skin over a period of time from about 8 hours to about 24 hours.

10. The method of claim 8, wherein the liquid organic substance is present from about 20% to about 70% by weight, or from about 10% to about 85% by weight.

11. The method of claim 8, wherein the aqueous composition consists essentially of glycerin and water.

12. The method of claim 8, wherein the liquid organic substance is glycerin, diglycerol, a polyethylene glycol, an effective glyceryl derivative, an effective glycol derivative, or a mixture thereof.

13. The method of claim 12, wherein the polyethylene glycol is polyethylene glycol 200, polyethylene glycol 300, polyethylene glycol 400 or polyethylene glycol 600.

14. The method of claim 8, wherein the aqueous composition further comprises a viscosity-enhancing or gelling agent at a concentration of about 0. 1% to about 8% by weight.

15. A method for ameliorating dark spots on facial skin comprising administering to facial skin of a human an aqueous composition, that is not a water-immiscible cream, ointment or lotion, consisting essentially of an effective amount of one or more nonvolatile, slowly absorbed, water-miscible, liquid organic substances, and water, wherein the liquid organic substance is present from about 4% to about 99.9% by weight, and wherein the liquid organic substance is slowly absorbed into the facial skin over a period of time for at least about 4 hours.

16. The method of claim 15, wherein the liquid organic substance is slowly absorbed into the facial skin over a period of time from about 8 hours to about 24 hours.

17. The method of claim 15, wherein the liquid organic substance is present from about 20% to about 70% by weight, or from about 10% to about 85% by weight.

18. The method of claim 15, wherein the aqueous composition consists essentially of glycerin and water.

19. The method of claim 15, wherein the liquid organic substance is glycerin, diglycerol, a polyethylene glycol, an effective glyceryl derivative, an effective glycol derivative, or a mixture thereof.

20. The method of claim 19, wherein the polyethylene glycol is polyethylene glycol 200, polyethylene glycol 300, polyethylene glycol 400 or polyethylene glycol 600.

21. The method of claim 15, wherein the aqueous composition further comprises a viscosity-enhancing or gelling agent at a concentration of about 0. 1% to about 8% by weight.

22. A method for increasing the elasticity and firmness of facial skin comprising administering to the facial skin of a human an aqueous composition, that is not a water-immiscible cream, ointment or lotion, consisting essentially of an effective amount of one or more nonvolatile, slowly absorbed, water-miscible, liquid organic substances, and water, wherein the liquid organic substance is present from about 4% to about 99.9% by weight, and wherein the liquid organic substance is slowly absorbed into the facial skin over a period of time for at least about 4 hours.

23. The method of claim 22, wherein the liquid organic substance is slowly absorbed into the facial skin surface over a period of time from about 8 hours to about 24 hours.

24. The method of claim 22, wherein the liquid organic substance is present from about 20% to about 70% by weight, or from about 10% to about 85% by weight.

25. The method of claim 22, wherein the aqueous composition consists essentially of glycerin and water.

26. The method of claim 22, wherein the liquid organic substance is glycerin, diglycerol, a polyethylene glycol, an effective glyceryl derivative, an effective glycol derivative, or a mixture thereof.

27. The method of claim 26, wherein the polyethylene glycol is polyethylene glycol 200, polyethylene glycol 300, polyethylene glycol 400 or polyethylene glycol 600.

28. The method of claim 22, wherein the aqueous composition further comprises a viscosity-enhancing or gelling agent at a concentration of about 0.1 % to about 8% by weight.

29. A method for promoting shiny and moist facial skin surface comprising administering to the facial skin of a human an aqueous composition, that is not a water-immiscible cream, ointment or lotion, consisting essentially of an effective amount of one or more nonvolatile, slowly absorbed, water miscible liquid organic substances, and water, wherein the liquid organic substance is present from about 4% to about 99.9% by weight, wherein the liquid organic substance is slowly absorbed into the facial skin over a period of time for at least about 4 hours.

30. The method of claim 29, wherein the liquid organic substance is slowly absorbed into the facial skin over a period of time from about 8 hours to about 24 hours.

31. The method of claim 29, wherein the liquid organic substance is present from about 20% to about 70% by weight, or from about 10% to about 85% by weight.

32. The method of claim 29, wherein the aqueous composition consists essentially of glycerin and water.

33. The method of claim 29, wherein the liquid organic substance is glycerin, diglycerol, a polyethylene glycol, an effective glyceryl derivative, an effective glycol derivative, or a mixture thereof.

34. The method of claim 33, wherein the polyethylene glycol is polyethylene glycol 200, polyethylene glycol 300, polyethylene glycol 400 or polyethylene glycol 600.

35. The method of claim 29, wherein the aqueous composition further comprises a viscosity-enhancing or gelling concentration of about 0. 1% to about 8 by weight.

36. A method for treating microorganisms on facial skin surface and for prophylaxis and treatment of facial acne comprising administering to the facial skin surface of a human an aqueous composition, consisting essentially of an effective amount of one or more nonvolatile, slowly absorbed, water miscible liquid organic substances, and water, wherein the liquid organic substance is present from about 4 to about 99.9% by weight, wherein the liquid organic substance is slowly absorbed into the facial skin over a period of time for at least about 4 hours.

37. The method of claim 36, wherein the liquid organic substance is slowly absorbed into the facial skin over a period of time from about 8 hours to about 24 hours.

38. The method of claim 36, wherein the liquid organic substance is present from about 20% to about 70% by weight, or from about 10% to about 85% by weight.

39. The method of claim 36, wherein the aqueous composition consists essentially of glycerin and water.

40. The method of claim 36, wherein the liquid organic substance is glycerin, diglycerol, a polyethylene glycol, an effective glyceryl derivative, an effective glycol derivative, or a mixture thereof.

41. The method of claim 40, wherein the polyethylene glycol is polyethylene glycol 200, polyethylene glycol 300, polyethylene glycol 400 or polyethylene glycol 600.

42. The method of claim 36, wherein the aqueous composition further comprises a viscosity-enhancing or gelling concentration of about 0.1% to about 8% by weight.
